(19) United States
(12) Patent Application Publication
(10) Pub. No.: US 2021/0183484 A1
(43) Pub. Date: Jun. 17, 2021

(54) HIERARCHICAL CNN-TRANSFORMER BASED MACHINE LEARNING

(71) Applicant: SURGICAL SAFETY TECHNOLOGIES INC., Toronto (CA)

(72) Inventors: Chantal SHAIB, Toronto (CA); Jinyue FENG, Toronto (CA); Frank RUDZICZ, Toronto (CA)

(21) Appl. No.: 17/114,184

(22) Filed: Dec. 7, 2020

Related U.S. Application Data
(60) Provisional application No. 62/944,649, filed on Dec. 6, 2019.

(51) Int. Cl. G16H 10/60; G06N 20/00; G06F 17/16

(52) U.S. Cl. CPC G16H 10/60; G06F 17/16; G06N 20/00

(57) ABSTRACT

Clinical prediction models often use structured variables and provide outcomes that are not readily interpretable by clinicians. Further, text medical notes may contain information not immediately available in structured variables. Applicants propose a hierarchical CNN-Transformer model with an explicit attention mechanism as an interpretable, multi-task clinical language model.

### CROSS-REFERENCE

This application is a non-provisional of, and claims all benefit to, U.S. Application No. 62/944,649, entitled “HIERARCHICAL CNN-TRANSFORMER BASED MACHINE LEARNING”, filed Dec. 6, 2019, incorporated herein by reference in its entirety.

### FIELD

Embodiments of the present disclosure generally relate to the field of machine learning, and more specifically, embodiments relate to devices, systems and methods for hierarchical convolutional neural network (CNN) transformer-based machine learning.

### INTRODUCTION

Clinical documentation, despite being potentially electronically captured, can be voluminous and difficult for electronic processing for insight extraction. In particular, text notes (e.g., free-text notes) contain valuable information but are impractical for human review due to the extremely large amount of time and effort required to manually process the notes.

Where the text notes are captured in electronically processable formats, such as notes processed with optical character recognition (OCR) or typed into a computer, the text notes can then be processed using various technical and computational approaches that automatically try to derive insights from what is captured in the notes.

However, many clinical decision support approaches do not take advantage of text notes due to the complex nature of clinical language and interpretation. Accordingly, important information stored in the text notes may simply be lost or not processed.

This can lead to adverse patient outcomes, for example, where an important detail in the text notes prepared by doctor are missing or not used in the output data structure from processing an electronic medical record.

Clinical prediction models can also use structured variables and provide outcomes that are not readily interpretable by clinicians. Further, text medical notes may contain information not immediately available in structured variables. Rules and specialized grammars can be applied to circumvent issues around clinical language, however these methods rely on the presence of certain phrases and spellings and do not account for the highly variable note structures across departments and hospitals.

Further, opaque deep learning models without explainability are often met with resistance in medical contexts.

### SUMMARY

Currently, there is no multi-task clinical language framework that jointly provides predictive outcomes and extracted rationales as explanation. To address some of the challenges, a multi-task language machine learning data model architecture is described herein that, in some embodiments, is adapted to provide indications representative of potential rationales for decisions in the medical context, where improved explainability is provided through the use of latent attention mechanisms. Embodiments of the proposed system were validated to conducted sepsis and mortality prediction on an example data set.

Natural language processing and explainable machine learning/artificial intelligence techniques are adapted to perform language-based clinical tasks and generate rationales for model decisions. In particular, the multi-task language machine learning data model architecture is adapted as a hierarchical CNN-Transformer model with an explicit attention mechanism as an interpretable, multi-task clinical machine learning data model architecture.

The machine learning data model architecture receives as inputs, data structures representing practitioner notes (e.g., strings, tokenized words, data structures storing ASCII/Unicode objects), and utilizes the hierarchical CNN-Transformer model which is trained to learn an importance of each unit of explanation, such as at the sentence or word level. The model can be utilized across a wide range of inputs and tasks, and more specific embodiments described herein are directed to use in a clinical setting (e.g., for long-form clinical text notes). The machine learning data model is an advanced deep learning model directed to natural language processing.

Multi-task aspects can be encoded through the use of structured data, in the form of structured variables, which can be used as another set of feature inputs for the machine learning data model architecture. These structured variables can include outputs from other systems or analyses converted into specific numerical data fields, and can include, for example, outputs from various clinical tests (e.g., complete blood count, blood pressure). For these, the structured variables can include for example, heart rates, oxygen saturation values, temperature values, among others. In another embodiment, the structured data includes structured variables extracted from machine learning prediction outputs, such as logits or normalized scores (e.g., predictive values extracted from video analysis, such as predictions associated with surgical skill, predictive values associated with whether a thermal injury event or bleeding event has occurred).

The approach of some embodiments utilizes a pre-trained-BERT (Bidirectional Encoder Representations from Transformers) to embed tokens into distributed representations, while overcoming the limitation of BERT on document length by using BERT as a static feature extractor on a sentence-by-sentence basis. The limitation of BERT on document length is particularly noted in relation to full text clinical notes, as these documents can have a large variation in length and an effective automated solution needs the ability to handle potentially very long document lengths.

In some embodiments, a convolutional neural network is built as an addition to BERT to extract sentence features to account for local n-gram contexts. The highly complex mechanisms of medical outcomes entail that the coexistence of other conditions may change the indication of another, so some embodiments apply a two-layer transformer encoder on top of sentence features to grasp the connections among descriptions. The approach includes hierarchical neural network structures tailored to process long documents (e.g., clinical documents >10 k words in length).

To obtain patient representation, as well as a source of explanations, the system is configured with a latent attention mechanism to dedicate a component of the model to explicitly learn the importance of each unit of explanation such as sentence or word. The units of explanation are sorted based on their corresponding attention weights and the top attended texts are selected as explanation. Depending on application contexts, the components of the model can be modularized and integrated in varying structure. For example, when processing long electronic medical records, a full model hierarchically captures the complicated information; however, a variant simpler model with word embeddings and latent attention can be used to processing short speech transcripts.

The machine learning system, after training, can be utilized to provide an automated mechanism to generate computer-based predictions of clinical outcomes such as sepsis and mortality solely based on text data, in some embodiments. The system can also be adapted, in another embodiment, to analyze characteristics of transcribed verbal communication in the operating room for aspects such as emotion and uncertainty, and generate corresponding computer-based predictions.

Furthermore, the system can also be adapted to provide rationales along with model predictions in clinical decision support contexts. These rationales can be generated, for example, by analyzing weights associated with particular subsections of the text (such as sentences, words, paragraphs, sections, etc.). In some embodiments, visual element characteristic modifications can be employed to show which particular subsections of the text, on a display interface or other type of output mechanism, were particularly highly weighted in respect of one or more predictions. In this example, the text can be highlighted by assigning color saturation levels based on the weighting.

In a first aspect, there is provided a system for conducting machine learning on text inputs having varying sequence lengths, the system comprising: a computer processor configured to: receive an input data set representing a text input relating to a healthcare event, split the input data set into n portions of tokens; provide the n portions into a transformer-based data model architecture to generate a transformer-based feature extractor data object X; provide the transformer-based feature extractor data object X into a convolutional neural network to obtain an n×d_{feature} matrix, S; apply a transformer-based encoder to obtain a matrix, S_{T}=Transformer(S) that shares the same dimension as S; determine a plurality of latent attention scores from the n portions using a neural network; based on the plurality of latent attention scores, generate one or more predicted classifications; and encapsulate the one or more predicted classifications as output data objects.

In another aspect, the varying sequence lengths include at least one sequence length greater than 512 tokens.

In another aspect, the n portions are at least one of n sentences, phrases, paragraphs, or other linguistic units.

In another aspect, the plurality of latent attention scores are incorporated into the output data objects.

In another aspect, a display interface is controlled based on the plurality of latent attention scores and a visual characteristic associated with each portion of the n portions of the input data set is modified based on a corresponding latent attention score of the plurality of latent attention scores.

In another aspect, the visual characteristic includes at least one of a size factor, a color factor, or an opacity factor.

In another aspect, the transformer-based data model architecture is a neural network maintained on computer memory.

In another aspect, the computer processor is further configured to conduct supervised training of the transformer-based data model architecture based on received data sets representative of evaluator classifications.

In another aspect, the output data objects are utilized to augment one or more electronic medical records with the one or more predicted classifications.

In another aspect, the output data objects include additional metadata representative of the latent attention scores such that the augmented one or more electronic medical records are adapted for improved explainability for the corresponding one or more predicted classifications.

In another aspect, wherein the latent attention scores are determined from the n portions of tokens using a position-wise feed-forward network, where given S_{T}, an n-dimensional vector αᵢₙₚᵤₜ is computed as:

aᵢₙₚᵤₜ=FeedForward(S_{T});

and an attention weight is determined using the relation:

a=Softmax(aᵢₙₚᵤₜ+aₘₐₛₖ);

wherein αₘₐₛₖ is a n-dimensional vector having values for unmasked positions and values for padding positions, and the attention weight is utilized to establish a n_{feature}-dimensional patient vector p that is computed as a weighted sum of the sentence features.

In another aspect, projection-weighted canonical correlation analysis is utilized to determine a correlation between learned textual features of transformer-based feature extractor data object X and one or more structured variable data.

In another aspect, the projection-weighted canonical correlation analysis (PWCCA) is utilized to normalize one or more importance weights that modify the attention weight corresponding to each of latent attention score of the plurality of latent attention scores.

In another aspect, a display interface is controlled based on the plurality of latent attention scores and a visual characteristic associated with each portion of the n portions of the input data set is modified based on a corresponding latent attention score of the plurality of latent attention scores.

In another aspect, the visual characteristic includes at least one of a size factor, a color factor, or an opacity factor.

In another aspect, the transformer-based data model architecture is a neural network maintained on computer memory, and wherein the computer processor is further configured to conduct supervised training of the transformer-based data model architecture based on received data sets representative of evaluator classifications.

In another aspect, the output data objects are utilized to augment one or more electronic medical records with the one or more predicted classifications.

In another aspect, the output data objects include additional metadata representative of the latent attention scores such that the augmented one or more electronic medical records are adapted for improved explainability for the corresponding one or more predicted classifications.

In another aspect, the additional metadata is used to cause rendered visual effects when a computer system renders the augmented one or more electronic medical records for display, the rendered visual effects including a modified visual characteristic controlled based at least on a corresponding latent attention score of the plurality of latent attention scores for each of the n portions of the input data set.

Corresponding methods and non-transitory computer-readable media storing machine interpretable instructions for execution on a processor to perform the methods are contemplated.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 is a block schematic diagram of an example system, according to some embodiments.

FIG. 2 is a plot showing the distribution of documents based on token lengths for the sepsis dataset, where 2842 out of 5147 documents exceed the token limits of BERT.

FIG. 3 shows example attention distribution over sentences in one patient document, according to some embodiments.

FIG. 4 is an illustrative visualization of Projection-Weighted Canonical Correlation Analysis (PWCCA).

FIG. 5 is an example test case output interface screen based on the BeerAdvocate dataset, according to some embodiments.

FIG. 6 is an example computing device that can be utilized for implementing the system of FIG. 1, according to some embodiments.

FIG. 7 is an example diagram showing extended structured variables associated with a machine learning data architecture adapted to track instrument usage, according to some embodiments.

FIG. 8 is a sample output from the machine learning data architecture adapted to track instrument usage, according to some embodiments.

### DETAILED DESCRIPTION

Electronic medical records (EMRs) store both structured data (e.g., vitals and laboratory measurements) and unstructured data (e.g., nursing and physician notes). Automated extraction of useful information in the unstructured data is particularly useful as clinical support systems rarely take advantage of free-text notes due to the complex nature of clinical language and interpretation. Rules and specialized grammars can be applied to circumvent issues around clinical language; however, these methods rely on the presence of certain phrases and spelling, and do not account for the highly variable note structures across departments and hospital.

Bidirectional Encoder Representations from Transformers (BERT) is a natural language processing model that can be used for language modelling. Bidirectional training can be contrasted with reviewing text sequences left to right or right to left. BERT mechanisms can be trained on text corpuses. BERT can be used for masking words in input and predictions for masked words, or estimating next sentences, among others. However, a technical limitation of BERT is in relation to sequence length (e.g., 512 tokens). Accordingly, BERT cannot be used directly as a fine-tuning language model on long documents.

Given the uniqueness of medical text, a combination of CNN and transformer encoders are proposed to capture phrase-level patterns and global contextual relationships. Additionally, latent attention layers are explored to generate rationales, potentially in combination with structured data as an additional input to yield improved multi-task latent attention scores for improved explainability. The system was validated using the MIMIC-III database in an example implementation to predict outcomes relating to sepsis and mortality in an intensive care unit.

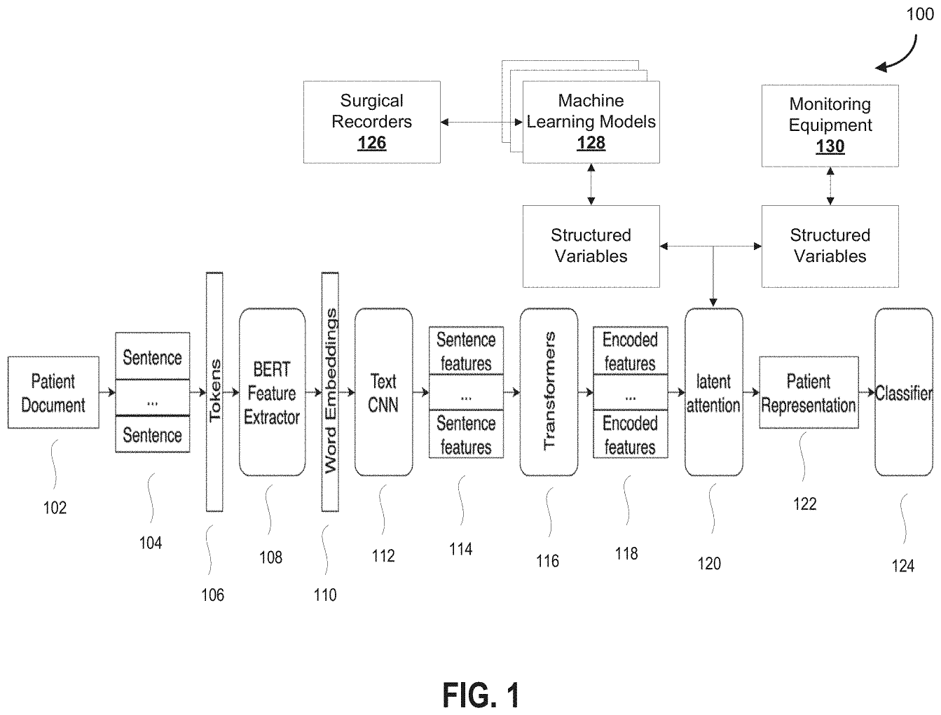

FIG. 1 is a block schematic diagram of an example system, according to some embodiments. The system 100, for example, can be a computer server including processors, computer memory, and data storage that operates alongside other electronic computing devices. The computing method of some embodiments can be affixed in the form of machine-interpretable instructions stored on non-transitory computer-readable media, the instructions, which when executed by a processor, cause the computer server to perform steps of a method. System 100 can reside in a data center and can be electronically coupled to informational systems that provide data sources for inputs 102, for example, across a message bus or other type of messaging interface.

The system 100, in some embodiments, is a clinical decision support tool that uses explanations to enhance model usability and reliability. Attention mechanisms provide plausible rationales for use in practice, even though it may not provide a complete internal representation of the model's behaviour, they may nonetheless be useful in promoting some level of explainability when automated predictions are revisited.

The system 100, in some embodiments, is adapted to append, for example, as associated metadata, one or more latent attention scores to be associated with individual text portions (e.g., sentences, word pairs) of various medical notes, as well as structured data (e.g., heart rate, blood pressure). In some embodiments, the structured data further includes machine processed outputs from one or more machine learning data model architectures that are adapted, for example, to record videos and/or audio of activities taking place in an operating theatre.

In these situations, system 100 may be adapted for multi-task learning, where notes are combined with attention scores associated with various output fields of the machine learning data model architectures, such as machine vision mechanisms for generating predictions of score values associated with surgeon competence, hand-washing quality, instrument usage, etc. Specific data values, such as an amount of tremor, fluidity, or jitter when using a scalpel, for example, can then be incorporated into the system 100 for generating the latent attention scores. Accordingly, these can be used as explainable outputs for assessing a prediction of an adverse outcome, such as sepsis or mortality.

The decision support mechanism of system 100 can be adapted to receive the latent attention scores and append them onto a data structure associated with a record of the procedure, such as an electronic medical record (EMR) or a performance tracking system (e.g., associated with particular healthcare practitioners).

The system 100 may be coupled to a display (e.g., a mainframe system) having a coupled display, which is configured to receive the latent attention scores (e.g., in a database or other type of linked data record), and utilize them as metadata in modifying how information is rendered or displayed to a user.

For example, specific notes in the EMR may have sentence fragments enlarged (e.g., larger font), in a different color, having a different opacity, extracted, etc., where the machine learning data model associated a particular entry with being highly contributing towards the prediction of the adverse outcome. This is helpful, for example, to identify medical errors (e.g., improper suturing or using of a thermal tool), medical operating procedures that could be improved (e.g., handwashing), among others. Furthermore, the system 100 can also be used to generate predictions (e.g., predict sepsis) before symptom onset allows for earlier intervention, thus improving patient outcomes.

System 100 is a computer implemented system that includes computing components adapted to receive as data set inputs, documents (e.g., patient documents 102 having sentences or other portions 104), which are then tokenized as tokens 106 for BERT feature extraction to establish word embeddings.

Pretrained BERT-based models can be used as static feature extractors to build layers upon the word embeddings to learn task-specific representations spanning long documents. Medical documents can easily contain thousands of tokens. With the sequence length limit of 512 tokens, using BERT as a fine-tuning language model on long documents is practically challenging or impossible. Instead, the problem can be approached in a depth-first manner and BERT can be used as a static feature extractor on a sentence-by-sentence basis.

These word embeddings 110 are then provided to a text convolutional neural network (CNN) 112, which extracts sentence features 114 for providing to transformers 116 that obtain encoded features. The system 100 includes a latent attention mechanism 120 that is trained over the encoded features 118 to learn an encoded representation of a patient.

The encoded patient representation 122 is provided to an overall classifier 124 that can be used to generate dataset outputs. These outputs can include data sets, such as arrays, linked lists, data objects or other data representations including indications or variables representative of predicted classifications.

Predicted classifications can include that this person may have various ailments (e.g., diabetes, sepsis), or physical characteristics (e.g., scoliosis, body type, height, weight), predispositions, among other classifications.

These classifications can be automatically extracted from the patient documents 102. The latent attention mechanism 120 may track weights associated with specific portions (e.g., sentences, words, n-grams) of the original patient document 102, which can be used to show on a display or an interface an estimated explain-ability associated with each classification (e.g., based on the weights afforded to each specific portion, an estimated importance can be determined, which can then be used to modify a visual factor on an interface, such as a color value, a size, a shading, among others).

A specific illustrative, non-limiting example is described in relation to example inputs 102, which include patient documents that undergo various levels of feature extraction to arrive at token-, sentence-, and patient-level representations. The explicit attention layer provides a latent representation 120 for a patient. The final, attended-to patient representation 122 is used in the classification task by the classifier 124.

In some embodiments, additional structured variables can be provided to the latent attention mechanism 120 as encoded features for updating the patient representation 122. The additional structured variables can represent multi-task inputs, and in some embodiments, may be inputs provided by various monitoring equipment 130, such as vital alert trackers, outputs from various clinical tests, as well as specific operation type information, such as a procedure type, among others. In a further embodiment, the additional structured variables include one or more normalized outputs from machine learning models 128, which, for example, may conduct automated analysis based on recordings obtained from surgical recorders 126. For example, surgical recorders 126 can include a “black box” recorder located in operating theatres that can include video cameras, audio recorders, biometric sensors, among others. The output data (e.g., video image frames) from the surgical recorders 126 can be automatically processed by the machine learning models 128 to generate various machine-generated prediction values, such as estimations of unwarranted types of injuries (e.g., thermal damage) or adverse events (e.g., bleeding), or quality scores associated with various events taking place during the operating theatre (e.g., sanitation score associated with quality of hand-washing).

These structured variables can be converted into or provided in the form of normalized numerical scores, which can then be provided to the system 100 to modify, for example, one or more weightings associated with the latent attention scores associated with each of the tokens. In some embodiments, latent attentions scores may also be determined for each of the structured variables themselves to provide for additional explainability as it relates to a potential contribution for a classification task or predictive task associated with a particular structured variable. For example, if a structured variable is associated with instrument jitter and the adverse output classified is an injury associated with the fine-ness of a surgical cut (e.g., during delicate intraocular surgery), a high latent attention score associated with the jitter score may be helpful for downstream analysis and establishing corrective actions.

As described in various embodiments herein, projection-weighted canonical correlation analysis (PWCCA) based approaches may be utilized to incorporate multi-task attention into the determination of various latent attention scores.

The development and validation of this solution requires expertise in deep learning, natural language processing (NLP), human-computer interaction, and medicine. From earlier experimental stages, clinical knowledge is required to derive valid and learnable datasets from the massive MIMIC III (Medical Information Mart for Intensive Care III) dataset. Both inputs and labels are obtained based on the nature of each clinical task as well as the requirements for training a deep model.

This solution implements natural language processing and explainable machine learning/artificial intelligence techniques to perform language-based clinical tasks and generate rationales for model decisions. In particular, the multi-task language machine learning data model architecture is adapted as a hierarchical CNN-Transformer model with an explicit attention mechanism as an interpretable, multi-task clinical machine learning data model architecture.

The approach of some embodiments utilizes a pre-trained-BERT (Bidirectional Encoder Representations from Transformers) to embed tokens into distributed representations, while overcoming the limitation of BERT on document length by using BERT as a static feature extractor on a sentence-by-sentence basis.

The limitation of BERT on document length is particularly noted in relation to full text clinical notes, as these documents can have a large variation in length and an effective automated solution needs the ability to handle potentially very long document lengths.

In some embodiments, a convolutional neural network is built as an addition to BERT to extract sentence features to account for local n-gram contexts. The highly complex mechanisms of medical outcomes entail that the coexistence of other conditions may change the indication of another, so some embodiments apply a two-layer transformer encoder on top of sentence features to grasp the connections among descriptions. The approach includes hierarchical neural network structures tailored to process long documents (e.g., clinical documents >10 k words in length).

Long documents are intrinsically challenging to process because of the size of the inputs and long-term contextual dependency. The approach proposed herein was adapted based on Applicants' experimentation on different levels from data processing practices to decision on structures of each layer.

Most applications of NLP methods in medical contexts adopted traditional methods such as bag-of-words or topic modelling and the limited deep learning methods are often deficient. Leveraging techniques such as transformers require expertise and experiments not achievable without sophisticated knowledge in the field of neural networks.

To develop and test the model as proposed herein, hands-on experience with deep learning frameworks and various python libraries was necessary. The evaluation of model explainability requires both knowledge about usability testing and clinical expertise. The evaluator needs to understand the generated rationales and assess the quality by performing diagnosis tasks. Without such intellectual resources, the solution would not be validated.

As described herein, a hierarchical CNN-Transformer model is proposed in system 100 with an explicit attention mechanism 120 as an interpretable, multi-task clinical language model.

Two tasks are chosen to evaluate the informativeness of the medical notes: sepsis and mortality prediction.

An evaluation protocol is outlined to evaluate model usability in a clinical decision support context. The relationship between the learned features of structured and unstructured variables is also explored using Projection Weighted Canonical Correlation Analysis. The model achieves an AUROC score of 0.75 on the sepsis task, and 0.78 on the mortality task. From domain-expert evaluations, Applicant finds that the model generates informative rationales that are promising in real-life application.

The multi-task model described herein in some embodiments leverages ClinicalBERT, which is a transformer-based language model that has been pre-trained on clinical corpora such as the Medical Information Mart for Intensive Care III (MIMIC-III; johnson2016mimic).

Given the uniqueness of medical text, Applicants introduce a combination of CNN 112 and transformer 116 encoders to capture phrase-level patterns on top of the base ClinicalBERT model. Additionally, the approach explores the utility of explicit linear attention layers for generating rationales.

Electronic medical records (EMRs) store both structured data (e.g., vitals and laboratory measurements) as well as unstructured fields (e.g., nursing and physician notes). Many clinical prediction tasks have focused on using structured data (e.g., desautels2016prediction, gultepe2013vital, ghassemi2014unfolding) which, despite their utility, may not capture all of the useful information in the unstructured data.

If Applicants are to make interpretable clinical decisions based on these two forms of data, Applicants must ensure that explainability is possible in both.

Here, Applicants use the MIMIC-III database to predict two outcomes: sepsis and mortality in the intensive care unit (ICU). Moreover, Applicants use canonical correlation analysis (CCA) to explore the relationships between the latent features learned from both structured and unstructured data. This solution can be beneficial for many scenarios.

First, it can automate the processing raw medical records in text form to populate variables in a medical database that would require manual labours which is time-consuming.

Second, it can be used as a real-life clinical decision support tool that identifies risks of undesirable patient outcomes from clinical notes that may not be immediately obvious to health-care providers. Also, the explanation feature helps direct users' attention to short fragments that are most relevant to a certain prediction from long medical notes, which may hugely improve efficiency.

Third, the solution can also be used for analysis of communications in clinical contexts, such as emotion detection in operating rooms. It allows fast and large-scale data processing that is not achievable by manual annotations.

Lastly, a model without interpretability may also help automate processes, but it would not allow human to easily assess the quality of model decisions that would naturally be subject to errors in complex medical tasks. The system functions to assist, not replace, clinical specialists in their work and provides an interface between human experts and AI models for such purpose.

The model can be trained on different datasets to perform different tasks. Similar data can benefit from pretrained weights that can be fine-tuned towards various downstream tasks. Hyper-parameters can be tuned to suit a specific task. Depending on the scope of a specific task, the individual modules can be combined or adjusted to match different levels of complexity.

In some embodiments, the approach utilizes a portion of the input 102 (e.g., a sentence) as a unit of explanation 104. In alternate embodiments, the unit can be changed to n-gram phrases or words. The attention scores can also be the base of more sophisticatedly engineered explanation generation algorithm. In a further embodiment, the system is configured for automatic summarization built upon the current model.

Structured data and text data complement each other. In some embodiments, the system 100 is configured for leveraging structured data and structured data may be used to augment the model and enhance model performance. A potential extension is to build a multi-view model with joint training objectives. Furthermore, in addition to uses for retrospective analysis, the system 100 can be implemented to process medical records in real-time to identify needs for early intervention. Additional modules can also be utilized to form an integral system that can perform more complicated functions, such as speech analysis in live operating rooms. Non-neural components can be incorporated to perform easier task such as keyword detection in texts, and a multi-pass system can thus be developed to efficiently deal with multiple aspects in large-scale applications.

System 100 can be utilized for different use cases, for example, in relation to expert analysts, where for the stakeholder, system 100 can help accelerate the assessment of patient files after discharge by suggesting relevant diagnoses, such as mortality or sepsis, and the corresponding rationale. System 100 can also be used to deploy non-technical skill training programs by automatically identifying communication instances with uncertainty or emotional content. Furthermore, system 100 can be deployed as an accessory to electronic medical note capture to provide a flagging system for patients who require earlier intervention.

Transformers have gained popularity in NLP given their downstream performance and parallelizability.

The success of transformer-based models such as BERT has inspired numerous studies to apply it to various domains. For example, BioBERT is pretrained on PubMed abstracts and articles, and outperforms BERT in biomedical tasks.

On tasks requiring specialized knowledge, such as biomedical named entity recognition, the base BERT model gave shorter or incorrect entity boundaries, whereas BioBERT was able to correctly identify biomedical entities and boundaries. Further refinements were made to BioBERT on the MIMIC-III dataset and the model released as ClinicalBERT.

To overcome the limitation of sequence length, the system 100 uses pretrained BERT models as static feature extractors and build layers upon the word embeddings to learn task-specific representations.

Explainable AI is an emerging field without a single methodology or set of evaluation metrics. The definition of model explainability also varies depending on the application context. An approach to language model explainability is through extractive rationales.

The wide application of attention mechanisms in NLP has led to an ongoing debate over whether attention can be used as explanation.

Applicants propose a clinical decision support tool and use explanations to enhance model usability and reliability. Therefore, Applicants adopt a view in that attention provides plausible rationales for model prediction even though it may not suffice as a faithful indicator of model behaviour.

### Clinical Tasks

Sepsis is a systemic inflammatory response to infection. If left untreated, sepsis can lead to life-threatening complications. The ability to predict sepsis before symptom onset allows for earlier intervention, thus improving patient outcomes. Previous work on sepsis detection has focused on both post-hoc identification as well as predicting the need for early intervention from structured data. As mortality has an explicit label in EMRs, the focus is on expiry likelihood for early intervention rather than post-hoc identification. Applicants focus on work that used the MIMIC-III database.

Insight used a predictive model that provided a score within a fixed-time window before the suspected onset of sepsis, on retrospective data. Similarly, an alternate approach proposed a method for identifying sepsis from vital signs; however, they did not account for time windows before sepsis onset, instead using only the last two available vital measurements before onset as the target. While these methods achieve robust results compared to traditional clinical measurements (e.g., qSOFA, SIRS), none take advantage of the unstructured data found in EMRs.

A further alternate approach used topic modelling for textual representations aggregated with structured patient data to predict mortality, but it was shown that using convolutional document embeddings for each patient outperformed such topic modelling strategies for mortality prediction.

Applicants deploy convolutional layers in the system 100 to obtain sentence level embeddings.

An alternate approach claimed that unstructured data in EMRs contain additional information not found in the structured variables. They used GloVe word embeddings to represent the notes for each patient, and only excluded discharge summaries to minimize explicit mentions of sepsis. Simply excluding discharge summaries, however, is not enough to avoid the label leakage problem. The diagnosis of sepsis may appear (directly or indirectly) in the notes as the clinician becomes aware of symptoms.

Applicants carefully filter notes to ensure no label leakage occurs. An alternate approach also found a modest improvement by using a combination of structured and unstructured variables, when compared to models that exclusively used one or the other, combined structured and unstructured data for sepsis prediction, using topic models and continuous-bag-of-words (CBOVV) to represent text. Despite success, GloVE word embeddings, topic models, and CBOW may not generally capture the complexity and contextual relationships between words in a given text.

To this end, system 100 implements a transformer-based model to represent the clinical notes.

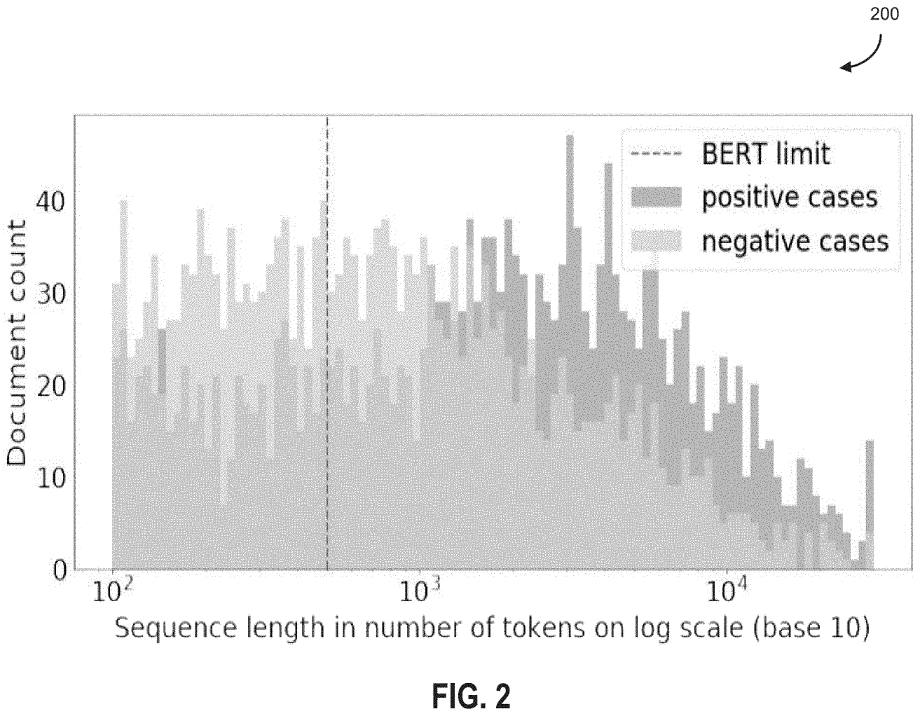

BERT and its variants have exhibited strong performance in various tasks; however, Applicants are interested in its application specifically in medical contexts. One limitation of BERT is that the maximum sequence length is 512 tokens, and thus using it as a fine-tuning language model on long documents is practically impossible. An alternate approach tackled the fixed-length context problem of transformers by incorporating segment recurrence mechanisms and proposed the Transformer-XL structure. XLNet outperformed BERT on benchmark tasks. However, it is still unrealistic to use such computationally consuming method on the dataset where patient records may contain thousands of tokens, as shown in FIG. 2.

FIG. 2 is a plot 200 showing the distribution of documents based on token lengths for the sepsis dataset, where 2842 out of 5147 documents exceed the token limits of BERT.

Instead, Applicants approach this problem in a depth-first manner and use BERT as a static feature extractor on a sentence-by-sentence basis. This feature-based approach with BERT has been proved to be nearly as effective as the fine-tuning approach in other tasks.

The system 100 splits each document into n sentences 104 of m tokens 106 and use a separate data loader with a sequential sampler to group them into sub-batches.

The input is truncated or padded at both the sentence- and token-level. The system 100 then feeds the sentences 104 into a BERT model 108 and take the mean of the last four encoder layers as token embeddings 110. [CLS] and [SEP] tokens can be omitted.

Given an input T=[t₁₁, t₂₁ . . . tₙ₁, t₁₂ . . . tₙₘ], where tᵢⱼ denotes the i^{th} token of the j^{th} sentence, the BERT feature extractor outputs

X=[x₁₁ . . . xₙₘ]=BERT(T), where xᵢⱼ is a d_{emb} dimensional vector (i.e., the hidden dimension of the BERT configuration) corresponding to tᵢⱼ.

Convolutional Layer

Previous studies using deep CNNs to process medical notes have achieved good results on tasks such as mortality prediction and ICD-9-CM diagnosis code classification. Specifically, a qualitative evaluation of text snippets from an attentional CNN indicated the model's ability to learn features that are deemed informative and diagnosis-relevant by a physician. This suggests that the CNN is suitable for extracting information regarding patient status at the phrase-level.

Deep CNNs use filters and pooling layers to learn features over a spatial domain in data. In NLP, this translates to learning n-gram or n-character features from textual data, where filter hyperparameters can be adjusted to produce different feature maps. For example, a feature map can search for patterns in two-word sequences or three-word sequences.

Given the sequential nature of CNN learning, features can provide contextual information between n-grams, however this is only limited to the filter bounds. CNNs provide a method for fast n-gram feature extraction, but do not capture any long-term dependencies between sequences outside of the filter (i.e. further along the sentence, or retroactively in the sequence).

The system 100, in some embodiments, uses CNNs because they are fast and have been shown to extract relevant, phrase-level features from medical notes. The system 100, in some embodiments, uses a convolutional layer followed by ReLU activation and 1D max-pooling to obtain sentence representations.

Taking X as input, the CNN outputs a n×d_{feature} matrix

S=MaxPool(ReLU(Conv(X)))
where d_{feature} is the number of output channels of the convolution layer.

Transformer Patient Encoder

Although other work in patient-clinician dialogue has explicitly used time-series information, medical notes frequently contain repeated segments of medical histories as well as plans for future treatment. Applicants assume the temporal sequence of patient conditions is already disrupted in the document so that the ability to learn time-series information is unnecessary.

Medical notes frequently contain repeated segments of medical histories as well as plans for future treatment, but the strict temporal order of patient conditions in clinical notes can be disrupted by repeating information, and the highly complex mechanisms of medical outcomes entail that the coexistence of other conditions may change the indication of another.

In BERT models, the contextual relationships among words are learned by the stacked transformer encoder layers. Similarly, the system 100, in some embodiments, applies a two-layer transformer encoder on top of sentence features to grasp the connections among descriptions.

Transformers excel in learning long-term contextual information surrounding a given token, especially when compared to CNNs. They are not constrained by only the information located in the filter boundaries; instead transformers are able to learn dependencies spanning over an entire sequence length. This is important because medical notes can contain information much further along in the text that can influence the interpretation of a given embedding.

Aggregating clinical notes for each patient results in documents so the approach overcomes the sequence length limitation of BERT by hierarchically learning compact representations of the relationship between sentences for a given patient document. Once the tokens are embedded, the CNN learns phrase level representations that are considerably shorter in sequence length than the token limitation for transformers.

Transformers rely on self-attention mechanism, instead of recurrent units as in RNN, to learn long-term dependencies. The gradients are more stable and the computations are parallel. For such reason, the model is easier and faster to train.

A two-layer transformer encoder is proposed on top of sentence features to capture a unified representation among descriptions.

This step of encoding results in a matrix, S_{T}=Transformer(S) that shares the same dimension as S.

Although multi-head attention is powerful, it is not yet clear how to derive rationales for model prediction using such an analysis. For model explainability, the system 100 instead applies an explicit attention mechanism 120 that is directly implementable and interpretable.

Latent Attention

The output of the transformer encoders are sentence-level features. To obtain patient representation, as well as a source of explanations, the system 100 uses a latent attention mechanism 120 adapted from latent attention for if-then program synthesis.

The goal of latent attention is to dedicate a component of the model to explicitly learn the importance of each unit of explanation such as sentence or word. In some embodiments, the latent attention described herein is also extended to structured variables in addition to the portions of the input text. In further embodiments, the incorporation of the structured variables as features is utilized to modify the latent attention scores associated with each of the portions of the input text.

The latent attention directly learns to weigh the importance of each sentence (and/or each structured variable) for a model prediction. The benefit of using latent attention is two-fold. First, as a pooling mechanism, the weighted sum of sentence features is more informative than the mean of sentence features. Second, it facilitates tracing back to each sentence based on the attention scores to obtain explanations.

The weighted sum of sentence features computed by latent attention layer is used as the patient representation for classification by a linear classifier.

The latent attention scores are determined from sentence features using a position-wise feed-forward network.

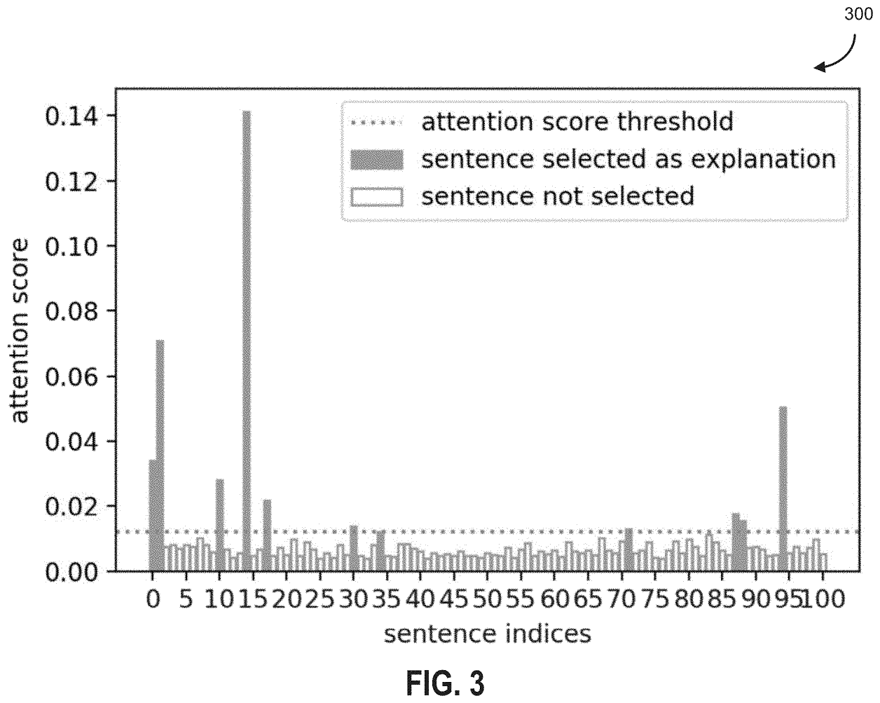

In FIG. 3, the attention scores are shown for each sentence. These scores come directly from the latent attention layer and help (e.g., by emphasizing via modified visual characteristics rendered on corresponding visual elements of an interface) determine which sentences are weighted more (i.e., more important) in the classification task.

Without this layer, it is difficult to interpret importance of sentences solely from the output of the transformer. Applicants found evidence that linear attention layers can provide meaningful values demonstrating the model's behaviour.

Given S_{T}, an n-dimensional vector aᵢₙₚᵤₜ is computed as aᵢₙₚᵤₜ=FeedForward(S_{T}) and the attention weight is a=Softmax(aᵢₙₚᵤₜ+aₘₐₛₖ), aₘₐₛₖ is an n-dimensional vector for which values unmasked positions are 0 and values at padding positions are −10000.

The final n_{feature}-dimensional patient vector p is computed at the weighted sum of sentence features as p=S_{T}·a and is fed into a linear layer and a softmax classifier.

Canonical Correlation Analysis

Classic canonical correlation analysis (CCA) provides a set of linear transformations that maximally correlate data points from multiple views. This is particularly useful when investigating the correlation between learned textual features and various structured data. This correlation can be utilized to establish comparisons between different neural network representations, and for determining how representations evolve over time.

Applicants propose using projection-weighted CCA (PWCCA) to explore the relationships between unstructured and structured features.

The structured variables are split into their respective clinical tests, shown in Table 1.

TABLE 1
Mapping of Clinical Tests to Corresponding Structured Variables

| Clinical Test | Related Structured Variable |
|---|---|
| Complete Blood | Hemoglobin Hematocrit; Mean Corpuscular |
| Count (CBC) | Hemoglobin; Platelets; Red Blood Cell Count; White |
| | Blood Cell Count |
| Prothrombin | Partial Thromboplastin Time; Prothrombin Time Inr; |
| Time (PT) | Prothrombin Time Pt |
| Urea, Creatinine, | Bicarbonate; Blood Urea Nitrogen; Chloride; |
| and Electrolytes | Creatinine; Potassium; Sodium |
| (UCE) | |
| Arterial Blood | Anion Gap; CO2 (etco2, pco2, etc.); Partial Pressure |
| Gases (ABG) | of Carbon Dioxide; pH |
| Blood Pressure | Central Venous Pressure; Diastolic Blood Pressure; |
| (BP) | Mean Blood Pressure; Pulmonary Artery Pressure |
| | Systolic; Systolic Blood Pressure |
| Individual | Glucose; Calcium; Calcium Ionized; Magnesium; |
| Tests (IND) | Phosphate; Phosphorous; Glascow Coma Scale Total |
| Pulmonary | Fraction Inspired Oxygen Set; Peak Inspiratory |
| Flowmetry (PF) | Pressure; Positive End-Expiratory Pressure Set; |
| | Respiratory Rate; Tidal Volume Observed |
| Primary | Heart Rate; Oxygen Saturation; Temperature |
| Vitals (PV) | |

In addition to providing explanations through sentence-level attention, Applicants use CCA to investigate the common information shared between learned textual features and various structured data categories. Specifically, Applicants examine the canonical components between two different feature representations, x and y, for each patient.

Given two vectors, x∈ [symbol] d×n and y∈ [symbol] d×m, where n and m denote the dimensional space of the unstructured and structured data respectively, the objective is:

$$( w 1 * , w 2 *) = arg {max}_{w 1 , w 2} \frac{w {1}^{′} KXYw 2}{\sqrt{w {1}^{′} KXXw 1 w {2}^{′} KYYw 2}} ,$$

Following the method of singular value CCA, Applicants use singular value decomposition to obtain the weights w₁, w₂. From this, Applicants obtained a total of min{n,m} canonical correlation coefficients.

The high dimensionality of the feature representations may result in noisy coefficients that hinder the similarity measurements.

Applicants use projection weighting to compute a weighted mean of the canonical variates, which accounts for the importance of CCA vectors relative to the original input. Understanding the correlated information between patient features from unstructured and structured data may provide insight on what latent information the model is learning from the text.

The PWCCA similarity between vectors x and y is computed with

$${d}_{pwcca} ( x , y ) = 1 - {\sum }_{c}^{i = 1} {α}_{i} {p}^{( i )}$$

where αᵢ denotes the normalized importance weights, and ρ⁽ⁱ⁾ the i^{th} CCA coefficient.

The PWCCA similarity can be utilized to establish correlation between learned textual features and various structured data. In some embodiments, the learned correlation can then be used to assign latent attention scores to the structured data, which can then be used to enhance the utility of a decision support tool (e.g., when reading machine learning outputs associated with a physician skill with instrument, certain scores, such as fluidity of movement, jitter, can be highlighted or noted during rendering to place increased visual emphasis). In another embodiment, the PWCCA similarity is utilized normalize one or more importance weights that modify the attention weight corresponding to each of latent attention score of the plurality of latent attention scores.

In some embodiments, the latent attention scores can also be utilized to modify a presentation order of various scores by re-ordering a presentation array (e.g., when providing feedback to a practitioner about his/her skills, the variables that contributed most to a finding of sepsis according to the correlated latent attention score can presented first). Similarly, these scores may be encapsulated as payloads to be stored on or alongside fields of an EMR such that downstream review or analysis can be utilized to elegantly provide inside on what latent information is learned.

Explanation Evaluation Protocols

Evaluating natural language systems remains a broad area of research. In addition to the task classification performance, Applicants outline an evaluation protocol that measures the quality of the extracted rationales by leveraging clinical-domain expertise.

For the following task, Applicants select at most 20 sentences as rationale in each patient record. More specifically, Applicants calculate a threshold score

$${a}_{threshold} = max ( \frac{1}{{n}_{s}} , {a}_{{sentence}_{i}} ) ,$$

where a denotes attention scores, nₛ is the number of sentences, and

$$i = min ( 20 , \frac{{n}_{s}}{10} ) .$$

FIG. 3 shows an example distribution 300 of attention scores and demonstrates the explanation generation criteria. To avoid overly complicated evaluation results, Applicants only include the correctly predicted cases. FIG. 3 shows example attention distribution over sentences in one patient document, according to some embodiments.

Labeling Task

In some embodiments, a display or other output mechanism is adapted by system 100 to provide (e.g., control rendering) of a display interface to present each sentence (e.g., sequentially) to physicians, who are instructed to identify whether a patient would develop sepsis or expire based on the texts. In further embodiments, the system 100 can record whether the human evaluator agrees with the model and the number of sentences presented until the label is concluded.

A test case fails if the evaluator is not able to make a decision by the end of the explanation. This method jointly evaluates i) whether the attended sentences are sufficient to provide useful clinical decision support, and ii) an appropriate threshold for how many sentences are needed. The recorded agreements or disagreements can be used for training.

Rating Task

In a second evaluation, Applicants sample cases not used in the labeling task. Applicants present the entirety of the rationale, including the task and label, to the evaluator. The evaluator is instructed to decide whether the sentence is useful for understanding or trusting the model decision. This method assesses whether attention scores are able to filter relevant sentences containing maximal information for the given task, as well as the usability of the model for the purpose of clinical decision support.

### Experiments

MIMIC-III

Medical Information Mart for Intensive Care III (MIMIC-Ill) is an open access clinical database comprising de-identified EMRs of 58,976 hospital admissions to the critical care units of the Beth Israel Deaconess Medical Center. Both structured and unstructured variables are recorded between 2001 and 2012. Note that, although ClinicalBERT is pretrained on MIMIC-Ill, this does not preclude its use from downstream tasks on the same dataset; alsentzer2019clinicalbert emphasize that any impact is negligible given the size of the entire MIMIC-III corpus compared to sub-sampled task corpora.

In this study, sepsis and mortality tasks were chosen because these are the standard tasks of this dataset. However, the machine learning model data architecture described herein is not necessarily specifically tailored to these tasks, and may be adapted to a wider range of potential applications.

Data Preprocessing

To avoid data leakage among hospital admissions of the same patient, Applicants only include patients with one hospital admission. Applicants then select adult patients from the single-admission group and obtain 31,245 hospital admissions as the base population.

For text preprocessing, Applicants removed punctuation (except periods and commas), masked identifiers, digits, single characters, and irrelevant information such as page headers. Applicants kept text preprocessing minimal and concatenated text from different note entries into one document for each patient.

The notes are appended in the order that they appear in the data and truncated to a maximum of 50,000 tokens after preprocessing.

For text, Applicants concatenated text from different note entries into one document for each patient and remove punctuation (except periods and commas), masked identifiers, digits, and single characters. When merging patients' notes, Applicants removed sentences that have already appeared in previous notes to avoid repetition. The notes are appended in chronological order according to their timestamps and truncated to a maximum of 50,000 tokens.

For mortality prediction, Applicants did not differentiate between note types.

For sepsis, Applicants include only nursing and physician notes as they have been identified as the most useful for the task by a physician.

After consulting with clinicians, Applicants excluded note types that are irrelevant to sepsis and select nursing and physician notes only. Whereas structured variables have explicit timestamps that can be easily related to symptom onset, the timestamp of a note may not. For example, a note containing descriptions of possible infection may be entered after antibiotic administration.

Anchoring notes with lab measurement timestamps significantly limits the number of positive cases in the dataset, especially when compared to other studies containing similar sepsis cohorts. Nonetheless, Applicants viewed the imposed time-window constraints as necessary to create an honest representation of prediction. Discharge summaries and any notes written after patient outcomes occurred are excluded to avoid direct access to the solution. Due to computational constraints, Applicants randomly sampled negative cases to balance and shrink the dataset.

For the structured data, Applicants use MIMIC-Extract to ensure a standard patient population. After obtaining time-binned cohort data, Applicants extracted measurements within the same time frames as the selected notes.

Sepsis

Each hospital admission is associated with a list of ICD-9-CM codes indicating diagnoses and procedures over the course of the patient's stay. ICD-9-CM codes can be unreliable, as they are assigned for billing purposes after the patient has been discharged. Thus, Applicants apply inclusion criteria based on the gold standard definition of sepsis.

Systemic inflammatory response syndrome (SIRS), characterized by abnormal body temperature, heart rate, respiratory rate, and white blood cell count, often precedes sepsis. In this task, Applicants aim to predict whether a patient in SIRS would become septic. This is in contrast to previous works, where the negative sepsis populations did not necessarily have SIRS.

Sepsis prediction from SIRS patients is a more restrictive task, as the model must learn features that are distinctive of sepsis onset rather than general indications of infection or SIRS. Applicants use ICD-9-CM codes to label cases, where patients with codes for explicit sepsis as well as the combination of infection and organ failure or SIRS are considered positive. Although ICD-9-CM codes can be unreliable, Applicants use multiple criteria to deal with false negatives and SIRS as a filter to avoid false positives. Applicants notice that very few notes are recorded before the first onset of SIRS possibly due to time delay in writing or logging notes. To compensate for the lack of data, notes before and within 24 hours of the first onset of SIRS are included. To avoid possible label leakage, certain sentences were removed (e.g., those containing mentions of sepsis or septic). The final cohort contain 1262 positive cases and 1500 negative cases.

In-ICU Mortality

In-ICU mortality is an explicit expiry timestamp in MIMIC-Ill; Applicants use this flag to identify the positive cohort for in-ICU mortality prediction. To ensure that negative samples also represent patient conditions in ICU units, Applicants only include notes written within ICU stays. The final dataset has 2562 positive cases and 2587 negative cases.

Clinical Vs Non-Clinical BERT

To compare the effect of pretraining BERT with domain-specific clinical data on the overall quality and performance of the model, Applicants substitute BioBERT and vanilla BERT as the token embedding component.

Applicants run both sepsis and mortality tasks on the different BERT models and compare the final performance. The results are shown in Table 2.

TABLE 2
Test performance scores using different BERT models

| | Sepsis | | | | Mortality | | | |
|---|---|---|---|---|---|---|---|---|
| Model | AUROC | F1 | Precision | Recall | AUROC | F1 | Precision | Recall |
| BERT | 0.72 | 69.3 | 64.3 | 75.0 | 0.75 | 74.2 | 77.7 | 70.9 |
| BioBERT | 0.72 | 71.2 | 59.8 | 88.1 | 0.76 | 76.8 | 72.6 | 81.6 |
| ClinicalBERT | 0.75 | 73.0 | 64.4 | 84.3 | 0.78 | 78.9 | 78.2 | 79.7 |

ClinicalBERT models converge significantly faster and outperform the other two models in both sepsis and mortality tasks. Unexpectedly, the BioBERT model performs worse than the base BERT model in the sepsis task. One possible explanation is that BioBERT is strongly shifted to the language in biomedical scientific literature, whereas clinical notes can be less technical or formal.

In comparing performance between tasks, the bioBERT and BERT models suffer more from a lack of in-domain pretraining in sepsis than mortality. This could be attributed to the highly clinical nature of describing sepsis-related complications when compared to the simpler language associated with mortality.

Considering the generated explanations, Applicants observe a pattern that mortality-predictive sentences are more likely to describe social interaction or behaviour of patients (e.g., family visits), and sepsis-predictive sentences tend to report clinical symptoms (e.g., changes in blood test results).

Canonical Correlation Analysis

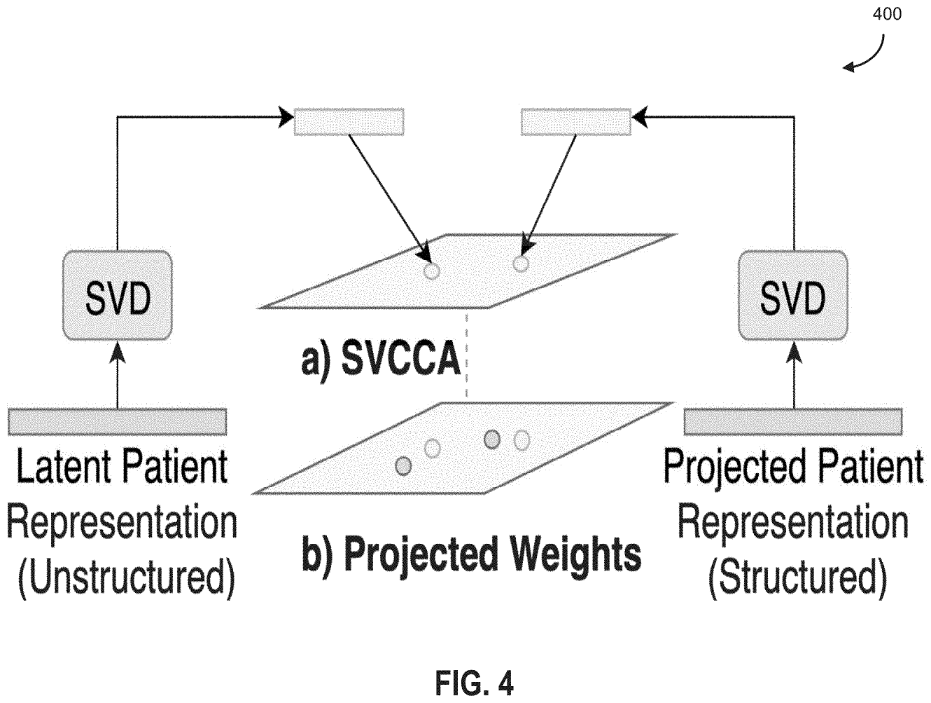

To investigate the relationships between patient features extracted from structured and text data, Applicants separately trained RNN models to learn representations from different groups (see Table 1) of laboratory measurements and conduct Projection-Weighted Canonical Correlation Analysis (PWCCA) (FIG. 4) to compute their similarities to patient features from the language model.

FIG. 4 is a visualization 400 of PWCCA. The patient representations are taken from the models before classifier. First, (a) a latent space is learned with Singular Vector Canonical Correlation Analysis (SVCCA), and then (b) The original representation is projected onto the learned latent space, and the PWCCA is computed.

Structured Data Model

To obtain a single vector from time-series structured data, Applicants construct a 2-layer single-directional GRU network followed by a linear layer to project the mean GRU output to a feature vector that has the same dimension as the language model feature vectors. Only the patients that appear in the language model are selected. Each model is trained for 50 epochs, and the best-performing one is used to extract features.

CCA Details

To avoid spurious correlation typically found in small datasets, the number of data points (nₛₐₘₚₗₑ) needs to be at least five times the feature dimension (d_{feature}). Therefore, Applicants include all shared patients between structured and unstructured datasets and over-sample the data for the sepsis task. Applicants set up random baselines for each test where Applicants randomly generate nₛₐₘₚₗₑ d_{feature}-dimensional vectors using the same sampling strategy as the real features. To ensure that the features are meaningful, Applicants only analyze features extracted by models that reach an AU ROC of at least 0.75.

It is important to note that Applicants constructed the structured dataset to obtain the patient representation, not to compare model performance. The structured inputs contain measurements after the onset of patient outcomes, so the metrics should not be compared to those of the language model.

Additionally, the structured data models failed to learn to predict sepsis from SIRS cohort, so Applicants included negative samples without SIRS whose data are extracted from random time frames.

Model performance and PWCCA similarity are listed in Table 3.

TABLE 3
Structured model test performance and
PWCCA similarity to text features.

| | |
|---|---|
| Sepsis | Mortality |

| Features | AUROC | Similarity | AUROC | Similarity |
|---|---|---|---|---|
| All | 0.75 | 0.68 | 0.92 | 0.762 |
| CBC | 0.77 | 0.80 | 0.5 | — |
| PT | 0.76 | 0.60 | 0.5 | — |
| UCE | 0.68 | — | 0.57 | — |
| ABG | 0.77 | 0.60 | 0.62 | — |
| BP | 0.76 | 0.65 | 0.5 | — |
| IND | 0.77 | 0.93 | 0.88 | 0.686 |
| PF | 0.78 | 0.61 | 0.62 | — |
| PV | 0.5 | — | 0.5 | — |
| Random | — | 0.45 | — | 0.361 |

The all category encompasses all test groups and their features. Only models reaching an AUROC higher than 0.75 are compared. See Table 1 for the full list of features and their corresponding test categories.

Feature Correlation

The similarity scores are subject to confounding factors such as noise and sample size. Due to limited data availability, Applicants can only comment on certain patterns.

The structured data model and language model converge to correlated solutions, compared to random baselines. Applicants do not observe any clear relationship between structured model performance and similarity, but the features learned from all lab measurements, which supposedly encode a more comprehensive patient representation than any subgroup alone, are close to the features learned from medical notes, especially in the mortality task. For the sepsis task, the test groups that are highly related to systematic inflammation or organ dysfunction (CBC, BP, IND) show especially strong correlation with the textual features. The results suggest that language models learn to encode the most relevant patient conditions for each outcome.

Evaluating Explanations

As Applicants have established, the purpose of model explainability is to assist humans in decision-making. An objective is a usable model that can be deployed as a real-life decision support tool. Therefore, Applicants focus on human evaluation as an assessment of rationale quality.

An objective is to provide a usable model that can be deployed as a real-life decision support tool. Therefore, there is a focus on human evaluation as an assessment of rationale quality. Applicants describe a novel evaluation approach that measures the quality of the extracted rationales by leveraging clinical domain expertise. Applicants avoid arbitrary judgements, and can work with the physician to tailor the definition of utility for each task. A stand-alone quantitative evaluation on non-clinical data of latent attention can be used as an explanation mechanism.

To obtain succinct meaningful explanations, the system can be configured to determine an attention threshold score

$${a}_{threshold} = max ( \frac{1}{{n}_{s}} , {a}_{{sentence}_{i}} ) ,$$

where α denotes attention scores, nₛ is the number of sentences, and i=min(20, nₛ/10). This ensures that selected sentences have higher attention scores than uniform attention and at most 10% of the original texts are included. To avoid burdening the evaluator, at most 20 sentences are selected for documents with more than 200 sentences. FIG. 3 shows example attention distribution generated over sentences in one patient document, and is an example distribution of attention scores and demonstrates the explanation generation criteria. Evaluation, for example, can be provided in the form of a system interacted through a command-line user interface.

Labeling Task

The labeling task is designed to evaluate the informativeness of the generated explanations.

Sentences are presented sequentially to an expert physician who chooses at each step to either predict patient outcome or check the next sentence. Sepsis has defined diagnosis criteria that must be followed in clinical practice, and information about such criteria are not necessarily available even in complete documents. However, mortality risk assessment, despite its difficulty, is common in critical care.

Therefore, Applicants only conducted the labeling task on the mortality dataset. Applicants compared human predictions to those of the model and note the number of selected sentences necessary for each prediction. A test case fails if the evaluator does not make a decision after reviewing all selected sentences. This method evaluates whether the attended sentences are sufficient to provide enough information for a clinical decision, and empirically evaluates the number of sentences needed for rationales.

The results are presented in Table 4. On average, the evaluator reaches a correct conclusion in mortality prediction 82.7% of the time by reading approximately 4 sentences per case (or a selected 0.5% of the note, on average). Such evidence strongly suggests that the model is capable of extracting the most relevant information from long documents. Applicants also observe a pattern that fewer sentences are needed for a correctly predicted case, which indicates that the ordering of sentences based on attention is generally reliable.

Such evidence strongly suggests that the system 100 model is capable of extracting the most relevant information from long documents. Applicants also observe a general pattern that less sentences are needed for a correctly predicted case, which indicates the ordering of sentences based on attention is reliable. In other words, sentences with the highest attention weights are the most predictive of patient outcomes.

TABLE 4
Labeling task results. Applicants list the number of cases,
percentage of concluded cases out of all cases, percentage
of correct cases out of total concluded cases, and the average
number of sentences read for both correct (c) and incorrect
(i) cases from top to bottom. These results are presented
separately for the positive and negative samples.

| | |
|---|---|
| Sepsis | Mortality |

| | | | | |
|---|---|---|---|---|
| Results | Pos | Neg | Pos | Neg |

| | | |
|---|---|---|
| N_{cases} | 119 | 136 |
| Conclusion | 98.4% | 98.5% |
| Correctness | 69.2% | 96.3% |
| Sentences Read (c) | 4.0 | 3.5 |
| Sentences Read (i) | 4.2 | 8.2 |

Interestingly, the evaluator almost correctly predicts all negative cases but not positive cases in the mortality task. Multiple reasons may account for the high false negative rate. First, mortality prediction is an intrinsically challenging task for humans.

A bias towards survival may naturally occur when a sentence can be interpreted differently based on various contexts. Second, explanations for negative cases are more likely to be independent from the contextual information that are not included in the rationales. A seemingly poor patient condition may translate to completely opposite outcomes depending on the coexistence of other conditions.

In real-life applications, providing full documents with highlighted explanations may be a useful solution that helps to direct users' attention to the most important parts without losing reference to additional contexts.

Rating Task

The rating task not only directly measures the usefulness of explanation at sentence-level, but simulates the scenario of model application.

In a second evaluation, Applicants sample cases not used in the labeling task. Applicants present model predictions and the entirety of the rationales sentence-by-sentence to an expert physician. The physician is instructed to decide whether each sentence in the rationale contains information that helps explain the model decision. To avoid arbitrary judgements, Applicants worked with the physician to develop clear definitions of explanation utility. This method assesses the average informativeness of selected sentences as well as the usability of the model for the purpose of clinical decision support.

Given the characteristics of mortality and sepsis, the evaluation is meaningful at the sentence- and caselevels for the two tasks. Compared to the labeling task, this evaluation is more prone to subjectivity. Therefore, Applicants use the labeling task as the proof of explanation quality, and the rating task as the demonstration of decision support in potential use cases. Table 5 summarizes the task results.

TABLE 5
Results of the rating task. Applicants provide the number
of sentences and the number of cases covered in each task.
The helpfulness score is an average score of all cases.

| | |
|---|---|
| Sepsis | Mortality |

| | | | |
|---|---|---|---|
| Pos | Neg | Pos | Neg |

| | | |
|---|---|---|
| N_{sentences} | 475 | 176 |
| N_{cases} | 40 | 20 |
| Helpfulness | 78.9% | 85.2% |

Table 5 summarizes the results. Between the positive and negative cases, an average of 72.2% of sentences in the mortality task and 86% of cases in the sepsis task are rated as helpful for understanding model decisions. A closer look at the results shows that 80% of the first four sentences are rated as helpful, which indicates that the specific algorithm that generates rationales could be refined to further exclude sentences with lower attention scores (see FIG. 3).

Conclusion

Language models can provide valuable support that can improve clinical decision-making. However, deep-learning-based NLP techniques have yet to be widely applied in the clinical domain. Applicants conduct a comprehensive set of studies to explore the many aspects of such an exciting field. Applicants also address challenges in extracting medical documents that are representative of a predictive task.

In system 100, Applicants leverage the power of domain-specific BERT and build a hierarchical CNN-Transformer model that can be potentially applied to any long-document processing tasks.

The model achieved AUROC scores of 0.75 and 0.78 on sepsis and mortality tasks. Applicants also addresses the growing interests in model explainability by experimenting with an effective linear attention mechanism.

Applicants emphasize the interaction between models and users in the design of explanation evaluation protocol. Applicants found that, not only are Applicants able to sufficiently predict cases with comparable performance to structured-only models, but Applicants are also able to provide useful rationales to support the prediction, as validated by medical domain-experts.

### APPENDIX

Validating Latent Attention as Explanation

As previously noted, the evaluation of language model explanations is not yet standardized. Despite the effort to make human evaluation fair and reliable, such qualitative measurements are still prone to bias and subjectivity.

To validate that latent attention can be used as an explanation, Applicants conduct a stand-alone experiment on the BeerAdvocate dataset. To Applicants' knowledge, this is the only dataset that has ground-truth annotations of sentences relevant to prediction results. Although the dataset is not crafted for the purpose of rationale evaluation, Applicants use it as a proxy to examine the quality of the attention scores.

The full BeerAdvocate dataset contains 1.5 million beer reviews describing four aspects (appearance, smell, palate, and taste), each corresponding to a rating on a scale of 0 to 5. There is a subset of 90 k reviews selected to minimize correlation between appearance and other aspects. In the experiment, Applicants use these 90 k reviews for training, and 994 annotated reviews for testing. The training set only has labels of ratings whereas the testing set has both labels of ratings and human annotations of sentence-level relevancy. Since all aspects have the exact same setups, it suffices to use appearance rating prediction as a proof of concept.

Applicants built a model BERT (pretrained base-cased model) and latent attention. Applicants fed static token embeddings from BERT 108 to the latent attention layer 120, which output sequence representations to be used for regression through a linear layer with sigmoid activation. Applicants train the model for 20 epochs and select the best performing one for testing.

In contrast to the clinical model, this model only attends to tokens and generates word-level explanations. For words separated by the WordPiece tokenizer, Applicants merged the tokens and average the attention weights. For each sentence, Applicants sorted the words based on their attention weights and take the top n words as the prediction rationale, where n equals to the total length of human-annotated sentences.

Applicants only use attention mechanisms without additional constraints, such as selection continuity, which makes the testing task even more challenging, as the annotations are ranges of words.

The model is evaluated according to mean squared error (MSE) and rationale precision

$${P}_{rationale} = \frac{{\sum }_{i = 1}^{N} \vert {S}_{t} ⋃ {A}_{i} \vert}{{\sum }_{t = 1}^{N} \vert {S}_{i} \vert} ,$$

where N is the number of test cases, y is the ground truth rating of appearance, ŷ is the predicted rating, Aᵢ is the set of word indices in the annotated covers, S is the set of word indices selected as model explanations, and |S|=|A|.

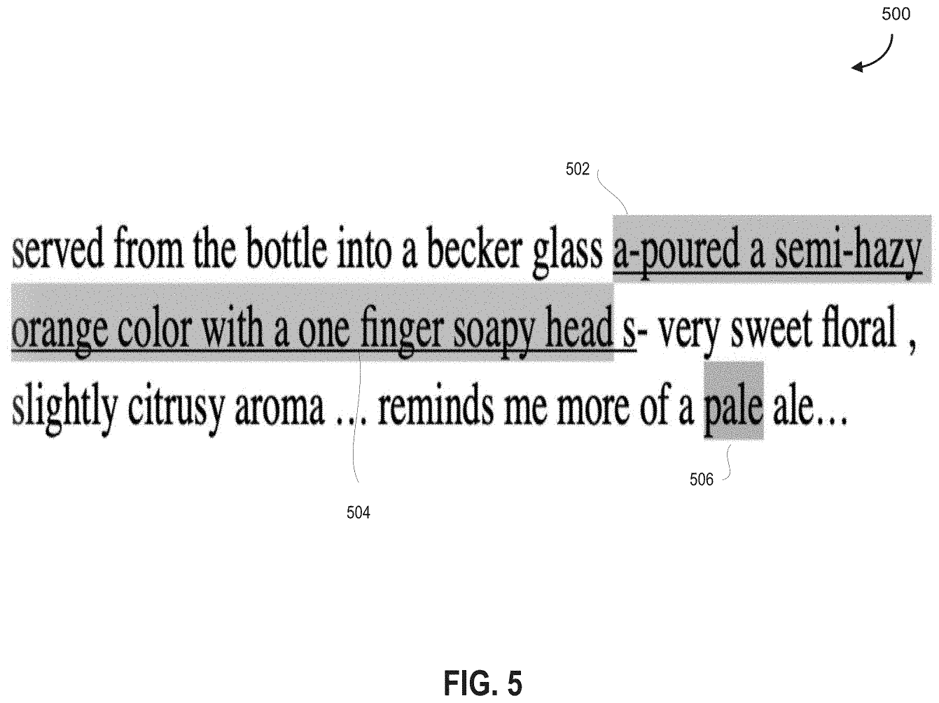

The system 100 reaches a rationale precision score of 76.39%, which indicates that the most attended words are mostly consistent with the annotations. FIG. 5 shows an example of appearance test results. FIG. 5 is an example test case output 500 based on the BeerAdvocate dataset.

In FIG. 5, the following display elements are modified to indicate various annotations (Blue background: attended tokens in annotation 502; Red background 506: attended tokens not in annotation 504; Underscore: annotation 506). Applicant notes that other types of annotations and visual characteristic modifications are possible.

The experiment demonstrates the usability of latent attention as an explanation mechanism.

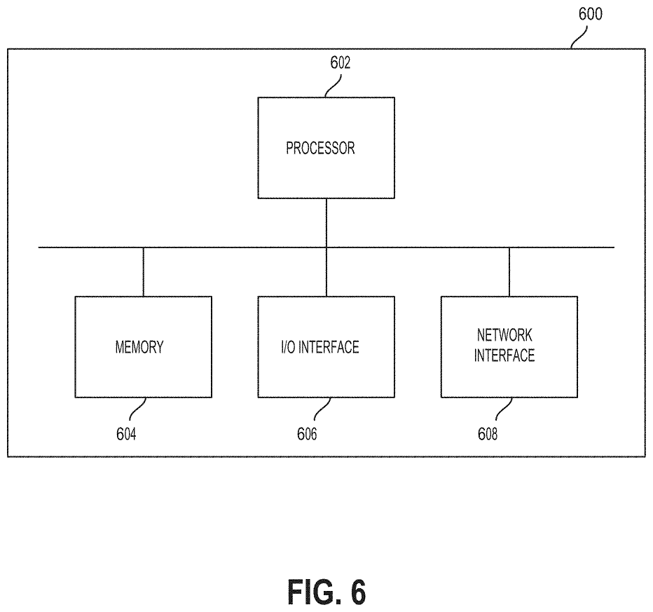

FIG. 6 is an example computing device 600 that can be utilized for implementing system 100, according to some embodiments. Device 600 includes a computer processor 602, which can be a processor adapted to receive and execute computational methods based on instructions loaded thereon in computer memory 604.

An input/output interface 606 is provided that can be adapted to receive inputs or provide output data sets, and a network interface 608 is provided that can be adapted to communicate input or output data sets, among other types of data messages. Device 600 can reside at a data center or be implemented using distributed computing resources, for example, in a cloud-based implementation that services multiple facilities (e.g., clinical facilities).

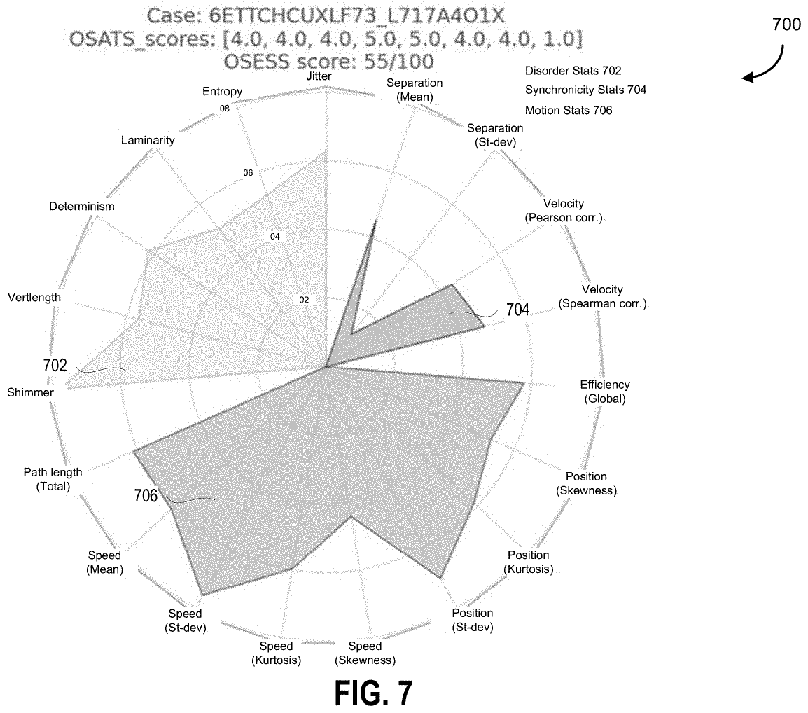

FIG. 7 is an example web diagram 700 showing extended structured variables associated with a machine learning data architecture adapted to track instrument usage, according to some embodiments. Other types of renderings are possible, and in some embodiments, the structured variables are stored in a data structure and input as encoded features for analysis.

Multi-task aspects can be encoded through the use of structured data, in the form of structured variables, which can be used as another set of feature inputs for the machine learning data model architecture. These structured variables can include outputs from other systems or analyses converted into specific numerical data fields, and can include, for example, outputs from various clinical tests (e.g., complete blood count, blood pressure). For these, the structured variables can include for example, heart rates, oxygen saturation values, temperature values, among others.

As described above, extended structured variables can include structured variables extracted from machine learning prediction outputs, such as logits or normalized scores (e.g., predictive values extracted from video analysis, such as predictions associated with surgical skill, predictive values associated with whether a thermal injury event or bleeding event has occurred).

In FIG. 7, a web diagram is shown as a pictorial representation of an example input structured data set that can be provided to the system 100. In FIG. 7, the web diagram shows a number of tracked instrumentation values that were generated from machine vision data obtained from surgical recorders 126.

In this example, the relevant image frames of video recordings were utilized to identify regions of interest (e.g., pixels) associated with instruments being utilized by the surgical practitioner. The regions of interest were then analyzed using object detection approaches to generate one or more structured variables associated with different features that could be extracted from the use of the instruments.

As shown in FIG. 7, disorder statistics 702 (e.g., jitter, entropy, laminarity, determinism, vertlength, shimmer), motion statistics 704 (e.g., separation, velocity), and synchronicity statistics 706 (e.g., speed, position, path lengths) can be tracked and represented either as raw structured variables, or in some embodiments, as normalized variables (e.g., normalized across a population set of surgeons).

While instrumentation values are shown in this example, other types of machine-learning derived structured data values are possible. For example, there may be structured data associated with predictive injuries, sanitation (e.g., hand washing quality), a predicted stress level (e.g., as associated with aural feature or biometric features), among others.

The subject matter of U.S. application Ser. No. 16/791,919, filed Feb. 15, 2019, and entitled “SYSTEM AND METHOD FOR ADVERSE EVENT DETECTION OR SEVERITY ESTIMATION FROM SURGICAL DATA” and U.S. application Ser. No. 16/881,906, filed May 22, 2020, and entitled “SYSTEM AND METHOD FOR SURGICAL PERFORMANCE TRACKING AND MEASUREMENT”, are incorporated herein by reference in their entireties.

These structured variables can be incorporated, for example, using SVCCA as indicated in FIG. 4 to establish projected weights for modifying one or more latent attention scores. In another embodiment, latent attention scores can be associated with the structured variables as well.

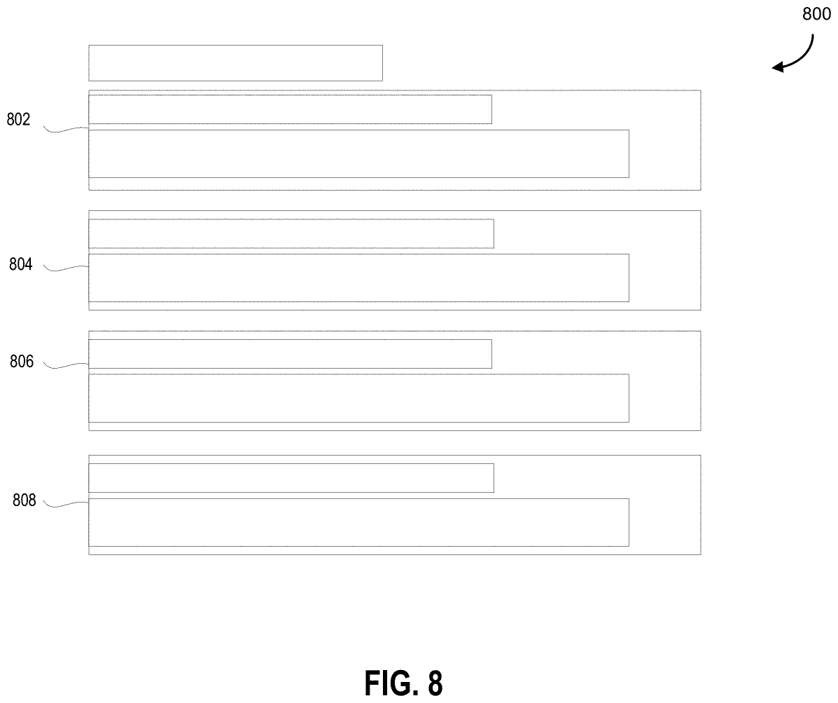

FIG. 8 is a sample output 800 from the machine learning data architecture adapted to track instrument usage, according to some embodiments. In FIG. 8, the normalized scores are shown for velocity (Spearman) at 802, velocity (Pearson) 804, separation (standard) at 806, and separation (mean) at 808. Each of these scores were normalized as percentile scores based on a population of surgeons, and can be utilized as input structured variables for system 100.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present application is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the claimed embodiments are intended to include within their scope such processes, machines, manufacture, compositions of matter, means, methods, or steps.

As can be understood, the examples described above and illustrated are intended to be exemplary only.

What is claimed is:

1. A system for conducting machine learning on text inputs having varying sequence lengths, the system comprising:
a computer processor configured to:
receive an input data set representing a text input relating to a healthcare event,
split the input data set into n portions of tokens;
provide the n portions into a transformer-based data model architecture to generate a transformer-based feature extractor data object X;
provide the transformer-based feature extractor data object X into a convolutional neural network to obtain an n×d_{feature} matrix, S, where S=MaxPool(ReLU(Conv(X))), and d_{feature} is a number of output channels of the convolutional neural network;
apply a transformer-based encoder to obtain a matrix, S_{T}=Transformer(S) that shares the same dimension as S;
determine, using at least S_{T}, a plurality of latent attention scores corresponding to each portion of the n portions using a neural network; and
encapsulate the plurality of latent attention scores as output data objects.

2. The system of claim 1, wherein the latent attention scores are determined from the n portions of tokens using a position-wise feed-forward network, where given S_{T}, an n-dimensional vector αᵢₙₚᵤₜ is computed as:
aᵢₙₚᵤₜ=FeedForward(S_{T});
and an attention weight is determined using the relation:
a=Softmax(aᵢₙₚᵤₜ+aₘₐₛₖ);
wherein αₘₐₛₖ is a n-dimensional vector having values for unmasked positions and values for padding positions, and the attention weight is utilized to establish a n_{feature}-dimensional patient vector p that is computed as a weighted sum of the sentence features.

3. The system of claim 2, wherein projection-weighted canonical correlation analysis is utilized to determine a correlation between learned textual features of transformer-based feature extractor data object X and one or more structured variable data.

4. The system of claim 3, wherein the projection-weighted canonical correlation analysis is utilized to normalize one or more importance weights that modify the attention weight corresponding to each of latent attention score of the plurality of latent attention scores.

5. The system of claim 1, wherein a display interface is controlled based on the plurality of latent attention scores and a visual characteristic associated with each portion of the n portions of the input data set is modified based on a corresponding latent attention score of the plurality of latent attention scores.

6. The system of claim 5, wherein the visual characteristic includes at least one of a size factor, a color factor, or an opacity factor.

7. The system of claim 1, wherein the transformer-based data model architecture is a neural network maintained on computer memory, and wherein the computer processor is further configured to conduct supervised training of the transformer-based data model architecture based on received data sets representative of evaluator classifications.

8. The system of claim 1, wherein the output data objects are utilized to augment one or more electronic medical records with the one or more predicted classifications.

9. The system of claim 8, wherein the output data objects include additional metadata representative of the latent attention scores such that the augmented one or more electronic medical records are adapted for improved explainability for the corresponding one or more predicted classifications.

10. The system of claim 9, wherein the additional metadata is used to cause rendered visual effects when a computer system renders the augmented one or more electronic medical records for display, the rendered visual effects including a modified visual characteristic controlled based at least on a corresponding latent attention score of the plurality of latent attention scores for each of the n portions of the input data set.

11. A method for conducting machine learning on text inputs having varying sequence lengths, the method comprising:
receiving an input data set representing a text input relating to a healthcare event, splitting the input data set into n portions of tokens;
providing the n portions into a transformer-based data model architecture to generate a transformer-based feature extractor data object X;
providing the transformer-based feature extractor data object X into a convolutional neural network to obtain an n×d_{feature} matrix, S, where S=MaxPool(ReLU(Conv(X))), and d_{feature} is a number of output channels of the convolutional neural network;
applying a transformer-based encoder to obtain a matrix, S_{T}=Transformer(S) that shares the same dimension as S;
determining, using at least S_{T}, a plurality of latent attention scores corresponding to each portion of the n portions using a neural network; and
encapsulating the plurality of latent attention scores as output data objects.

12. The method of claim 11, wherein the latent attention scores are determined from the n portions of tokens using a position-wise feed-forward network, where given S_{T}, an n-dimensional vector αᵢₙₚᵤₜ is computed as:
aᵢₙₚᵤₜ=Feedforward(S_{T});
and an attention weight is determined using the relation:
a=Softmax(aᵢₙₚᵤₜ+aₘₐₛₖ);
wherein αₘₐₛₖ is a n-dimensional vector having values for unmasked positions and values for padding positions, and the attention weight is utilized to establish a n_{feature}-dimensional patient vector p that is computed as a weighted sum of the sentence features.

13. The method of claim 12, wherein projection-weighted canonical correlation analysis is utilized to determine a correlation between learned textual features of transformer-based feature extractor data object X and one or more structured variable data.

14. The method of claim 13, wherein the projection-weighted canonical correlation analysis is utilized to normalize one or more importance weights that modify the attention weight corresponding to each of latent attention score of the plurality of latent attention scores.

15. The method of claim 11, wherein a display interface is controlled based on the plurality of latent attention scores and a visual characteristic associated with each portion of the n portions of the input data set is modified based on a corresponding latent attention score of the plurality of latent attention scores.

16. The method of claim 15, wherein the visual characteristic includes at least one of a size factor, a color factor, or an opacity factor.

17. The method of claim 11, wherein the transformer-based data model architecture is a neural network maintained on computer memory, and wherein the computer processor is further configured to conduct supervised training of the transformer-based data model architecture based on received data sets representative of evaluator classifications.

18. The method of claim 11, wherein the output data objects are utilized to augment one or more electronic medical records with the one or more predicted classifications.

19. The method of claim 18, wherein the output data objects include additional metadata representative of the latent attention scores such that the augmented one or more electronic medical records are adapted for improved explainability for the corresponding one or more predicted classifications.

20. A non-transitory computer readable medium storing machine interpretable instructions, which when executed by a computer processor, cause the computer processor to perform steps of a method for conducting machine learning on text inputs having varying sequence lengths, the method comprising:
receiving an input data set representing a text input relating to a healthcare event,
splitting the input data set into n portions of tokens;
providing the n portions into a transformer-based data model architecture to generate a transformer-based feature extractor data object X;
providing the transformer-based feature extractor data object X into a convolutional neural network to obtain an n×d_{feature} matrix, S, where S=MaxPool(ReLU(Conv(X))), and d_{feature} is a number of output channels of the convolutional neural network;
applying a transformer-based encoder to obtain a matrix, S_{T}=Transformer(S) that shares the same dimension as S;
determining, using at least S_{T}, a plurality of latent attention scores corresponding to each portion of the n portions using a neural network; and
encapsulating the plurality of latent attention scores as output data objects.
